(19) United States
(12) Patent Application Publication
(10) Pub. No.: US 2011/0053935 A1
(43) Pub. Date: Mar. 3, 2011

(54) FUSED PYRIDINES ACTIVE AS INHIBITORS OF C-MET

(76) Inventors: Adrian John Folkes, Basel (CH); Paul Goldsmith, Basel (CH); Neil Anthony Pegg, Basel (CH)

(21) Appl. No.: 12/864,112

(22) PCT Filed: Jan. 26, 2009
(86) PCT No.: PCT/GB09/00211

§ 371 Date: Nov. 19, 2010

(30) Foreign Application Priority Data
Jan. 25, 2008 (GB) 0801416.9

(51) Int. Cl. A61K 31/4709; C07D 401/04; C07D 413/14; C07D 417/14; C07D 401/14; A61K 31/5377; A61K 31/496; A61K 31/506; A61P 35/00; A61P 9/00; A61P 37/00; A61P 27/02

(52) U.S. Cl. 514/235.2; 546/167; 544/128; 544/363; 544/331; 514/314; 514/253.06; 514/275

(57) ABSTRACT

Fused pyridines of formula (I) and the pharmaceutically acceptable salts thereof have activity as inhibitors of c-Met and may thus be used to treat various diseases and disorders including cancer. Processes for synthesizing the compounds are also described.

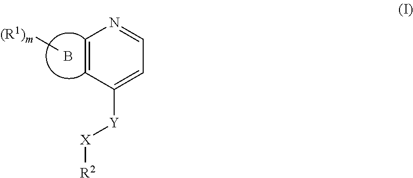

### FIELD OF THE INVENTION

The present invention relates to fused pyridines and their use as inhibitors of c-Met.

### BACKGROUND TO THE INVENTION

The study of signal transduction pathways in normal and pathological states is of considerable interest because of the potential therapeutic benefit arising from new molecular agents targeting certain of these pathways associated with disease. Receptor tyrosine kinases (RTKs) are key enzymes in signal transduction pathways that catalyse the autophosphorylation of tyrosine residues within the cytosolic, C-terminal domain of the protein. This generates docking sites for the recruitment of downstream proteins and the subsequent propagation of signals involved in an array of cellular events including growth, proliferation and survival. More generally deregulated kinase signalling is implicated in a diverse range of pathological states including immunological and inflammatory disorders, cardiovascular and neurodegenerative disease. The known receptor tyrosine kinases encompass 20 families and many are oncogenes (Blume-Jensen P et al. 2001. Nature 411 355-365).

c-Met is the prototypic member of a subfamily of RTKs which includes the related proteins Ron (macrophage-stimulating protein receptor) and its chicken orthologue, Sea. The endogenous ligand is the growth and motility factor hepatocyte growth factor (HGF, also known as Scatter Factor). c-Met and HGF are expressed in a range of tissue types although their expression is normally restricted to cells of epithelial and mesenchymal origin. In contrast, tumour cells often express constitutively activated c-Met.

There is now a growing body of compelling evidence from both animal studies and cancer patients that HGF-Met signalling plays an important role in the development and progression of malignancy and is associated in particular with invasive phenotypes. c-Met and HGF are highly expressed relative to surrounding tissue in numerous cancers and their expression correlates with poor patient prognosis (Jiang, W et al. 1999 Crit. Rev. Oncol.-hematol., 29, 209-248.) Activating point mutations in the kinase domain of c-Met are implicated in the cause of sporadic and hereditary forms of papillary renal carcinoma (Danilkovitch-Miagkova, A et al 2002. 1 J. Clin. Invest. 109, 863-867). c-Met is a marker for both cancer and malignancy and agents that inhibit c-Met-HGF signalling can be expected to ameliorate disease progression in relevant cancers.

### SUMMARY OF THE INVENTION

It has now been found that a novel class of fused pyridines are effective inhibitors of c-Met. Accordingly, the present invention provides a compound for use as an inhibitor of c-Met, which compound is a fused pyridine of formula (I):

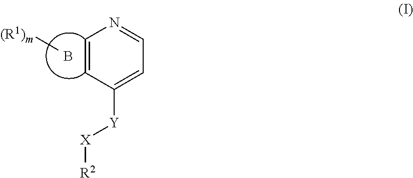

wherein
B is an aryl or heteroaryl ring;
each R¹, which are the same or different when m is greater than 1, is selected from H, halogen, CN, OR³, alkyl, alkenyl, alkynyl, CF₃, —O(C(R³)₂)ₙNR⁴R⁵, —NR³(C(R³)₂)ₙNR⁴R⁵, —NR⁴R⁵, —CONR⁴R⁵, —SO₂NR⁴R⁵, NO₂, —S(O)ₚR³, —CO₂R³, —NR³COR³, —NR³SO₂R³ and R⁶;
m is 0, 1 or 2;
Y is a heteroaryl, dihydroheteroaryl or aryl ring, or a group —C≡C—C(R′)₂—;
X is selected from —C(R³)₂—, —O—, —S—, —SO—, —SO₂—, —NR³—, —NR³SO—, —NR³SO₂—, —N(SO₂R³)—, —CO—, —CONR³—, —NR³CO—, —NR³CONR³—, —CSNR³—, —NR³CS— and —NR³CSNR³;
R² is selected from aryl which is unsubstituted or substituted, heteroaryl which is unsubstituted or substituted, C₁-C₆ alkyl, C₁-C₆ alkenyl and C₁-C₆ alkynyl;
R³ is selected from H, C₁-C₆ alkyl, C₁-C₆ alkenyl and C₁-C₆ alkynyl;
R⁴ and R⁵, which are the same or different, are each selected from H, C₁-C₆ alkyl, C₂-C₆ alkenyl and C₂-C₆ alkynyl, or R⁴ and R⁵ together with the N atom to which they are attached form a 5- or 6-membered heterocyclic ring containing 0, 1 or more additional heteroatoms selected from N, O and S;
n is 2 or 3;
p is 1 or 2;
R⁶ is an aryl or heteroaryl ring which is unsubstituted or substituted; and
R′ is H or C₁-C₆ alkyl;
   or a pharmaceutically acceptable salt thereof.

The invention also provides:

the use, in the manufacture of a medicament for use as an inhibitor of c-Met, of a compound which is a fused pyridine of formula (I) as defined above, or a pharmaceutically acceptable salt thereof; and

a method of treating a patient in need of an inhibitor of c-Met, which method comprises administering to the patient a compound which is a fused pyridine of formula (I) as defined above, or a pharmaceutically acceptable salt thereof.

Most of the fused pyridines of formula (I) are novel. Accordingly, the present invention further provides a compound which is a fused pyridine of formula (I′):

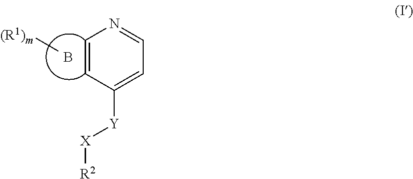

wherein
B is an aryl or heteroaryl ring;
each R¹, which are the same or different when m is greater than 1, is selected from H, halogen, CN, OR³, alkyl, alkenyl, alkynyl, CF₃, —O(C(R³)₂)ₙNR⁴R⁵, —NR³(C(R³)₂)ₙNR⁴R⁵, —NR⁴R⁵, —CONR⁴R⁵, —SO₂NR⁴R⁵, NO₂, —S(O)ₚR³, —CO₂R³, —NR³COR³, —NR³SO₂R³ and R⁶;
m is 0, 1 or 2;
Y is a heteroaryl or dihydroheteroaryl ring, or a group —C≡C—C(R′)₂—;
X is selected from —O—, —S—, —SO—, —SO₂—, —NR³—, —NR³SO—, —NR³SO₂—, —N(SO₂R³)—, —CO—, —CONR³—, —NR³CO—, —NR³CONR³—, —NR³CS— and —NR³CSNR³, provided that X is other than —NH— when Y is a pyrimidine ring;
R² is selected from aryl which is unsubstituted or substituted, heteroaryl which is unsubstituted or substituted, C₁-C₆ alkyl which is unsubstituted, and C₂-C₆ alkenyl and C₂-C₆ alkynyl which are unsubstituted or substituted;
R³ is selected from H, C₁-C₆ alkyl, C₂-C₆ alkenyl and C₂-C₆ alkynyl;
R⁴ and R⁵, which are the same or different, are each selected from H, C₁-C₆ alkyl, C₂-C₆ alkenyl and C₂-C₆ alkynyl, or R⁴ and R⁵ together with the N atom to which they are attached form a 5- or 6-membered heterocyclic ring containing 0, 1 or more additional heteroatoms selected from N, O and S;
n is 2 or 3;
p is 1 or 2;
R⁶ is an aryl or heteroaryl ring which is unsubstituted or substituted; and
R′ is H or C₁-C₆ alkyl;
   or a pharmaceutically acceptable salt thereof.

### DETAILED DESCRIPTION OF THE INVENTION

An alkyl group is a straight or branched chain saturated hydrocarbon radical which is unsubstituted or substituted. Typically it is C₁-C₂₀ alkyl, for instance C₁-C₁₀ alkyl, such as C₁-C₆ alkyl. A C₁-C₆ alkyl group is linear or branched. A C₁-C₆ alkyl group is typically a C₁-C₄ alkyl group, for example a methyl, ethyl, n-propyl, i-propyl, n-butyl, sec-butyl or tert-butyl group. A C₁-C₆ alkyl group is unsubstituted or substituted, typically by one or more groups Z or R⁷ as defined below.

Z is selected from H, unsubstituted C₁-C₆ alkyl, halo, —OR, —SR, —(C(R⁸)₂)_{q}R, —CH₂OR, —CF₃, -(halo)-C₁-C₆ alkyl, —(C(R⁸)₂)_{q}O-(halo)-C₁-C₆ alkyl, —CO₂R, —(C(R⁸₂)_{q}CO₂R, —(C(R⁸)₂)_{q}COR, CF₂OH, CH(CF₃)OH, C(CF₃)₂OH, —(CH₂)_{q}OR, —(C(R⁸)₂)_{q}OR, —(CH₂)_{q}NR₂, —(C(R⁸)₂)_{q}NR₂, —C(O)N(R)₂, —(C(R⁸)₂)_{q}CONR₂, —NR₂, —(C(R⁸)₂)_{q}NR₂, —(C(R⁸)₂)_{q}NRC(O)R, —(C(R⁸)₂)_{q}NRC(O)OR, —S(O)ₚR, —S(O)ₚN(R)₂, —(C(R⁸)₂)_{q}S(O)ₚN(R)₂, —OC(O)R, —(C(R⁸)₂)_{q}OC(O)R, —OC(O)N(R)₂, —(C(R⁸)₂)_{q}OC(O)N(R)₂, —NRS(O)ₚR, —(C(R⁸)₂)_{q}NRS(O)ₚR, —NRC(O)N(R)₂, —(C(R⁸)₂)_{q}NRC(O)NR)₂, CN, —NO₂, ═O, a 5- to 12-membered aryl or heteroaryl group, which group is unsubstituted or substituted and a 4- to 7-membered saturated N-containing heterocyclic ring, wherein each R is independently selected from H, C₁-C₆ alkyl, C₃-C₁₀ cycloalkyl and a 5- to 12-membered aryl or heteroaryl group, the group being unsubstituted or substituted, or when two groups R are attached to an N atom they form, together with the N atom, a 4- to 7-membered saturated N-containing heterocyclic ring; p is 1 or 2 and q is 0, 1 or 2.

R⁷ is selected from C₁-C₆ alkoxy, OR⁸, SR⁸, S(O)ₚR⁸, nitro, CN, halogen, —C(O)R⁸, —CO₂R⁸, —C(O)N(R⁸)₂ and —N(R⁸)₂. R⁸, each of which is the same or different when more than one is present in a given substituent, is selected from H, C₁-C₆ alkyl and C₃-C₁₀ cycloalkyl, and p is 1 or 2. Typically R⁸ is H or C₁-C₆ alkyl.

A halogen or halo group is F, Cl, Br or I. Preferably it is F, Cl or Br. A C₁-C₆ alkyl group substituted by halogen may be denoted by the term “halo-C₁-C₆ alkyl”, which means an alkyl group in which one or more hydrogens is replaced by halo. A halo-C₁-C₆ alkyl group preferably contains one, two or three halo groups. A preferred example of such a group is trifluoromethyl.

A C₁-C₆ alkoxy group is linear or branched. It is typically a C₁-C₄ alkoxy group, for example a methoxy, ethoxy, propoxy, i-propoxy, n-propoxy, n-butoxy, sec-butoxy or tert-butoxy group. A C₁-C₆ alkoxy group is unsubstituted or substituted, typically by one or more groups Z or R⁷ as defined above.

An alkenyl group is an unsubstituted or substituted, straight or branched chain hydrocarbon radical having one or more double bonds. Typically it is C₂-C₈ alkenyl, for instance C₂-C₆ alkenyl, such as allyl, butenyl, butadienyl, pentenyl or hexenyl. When the alkenyl group is substituted it is typically substituted by one or more groups Z or R⁷ as defined above, or by alkyl which is unsubstituted or substituted by one or more groups Z or R⁷ as defined above.

An alkynyl group is an unsubstituted or substituted, straight or branched chain hydrocarbon radical having one or more triple bonds. Typically it is C₂-C₈ alkynyl, for instance C₂-C₆ alkynyl, such as ethynyl, propynyl or butynyl. When the alkynyl group is substituted it is typically substituted by one or more groups Z or R⁷ as defined above, or by alkyl which is unsubstituted or substituted by one or more groups Z or R⁷ as defined above.

An aryl group is a 5- to 12-membered aromatic carbocyclic group. It is monocyclic or bicyclic. Examples include benzene and naphthalene rings, which are present as phenyl and naphthyl groups. The group is unsubstituted or substituted, for instance by a group Z or R⁷ as defined above. Bicyclic aryl groups comprise an aromatic ring fused to a saturated, partially unsaturated ring, or to an aromatic carbocyclic ring. Examples of aryl groups include phenyl, naphthyl, anthracenyl, biphenyl, indenyl, indanyl, 1,2-dihydronaphthyl and 1,2,3,4-tetrahydronaphthyl groups. An aryl group is unsubstituted or substituted with one or more substituents, for instance by one, two or three substituents. Suitable substituents include groups Z or R⁷ as defined above.

A heteroaryl group is a 5- to 12-membered aromatic heterocyclic group which contains 1, 2, 3, or 4 heteroatoms selected from O, N and S. It is monocyclic or bicyclic. Typically it contains one N atom and 0, 1, 2 or 3 additional heteroatoms selected from O, S and N. It may be, for example, a 5- to 7-membered heteroaryl group. Examples of a heteroaryl group include pyrrole, pyrazole, triazole, tetrazole, indazole, thiazole, isothiazole, oxazole, isoxazole, indole, isoindole, 1,3-dihydro-indol-2-one, pyridin-2-one, pyridine, pyridin-3-ol, imidazole, 1,3-dihydro-benzimidazolone, benzimidazole, benzothiazole, benzothiadiazole, benzofuran, cinnolinyl, quinoline, isoquinoline, quinoxaline, quinazoline, pyrazolopyridine, aminopyrazolinone, imidazopyridine, pyrimidine, pyridazine, pyrazine and isatin groups.

In one embodiment the fused pyridine is of the following formula (Ia):

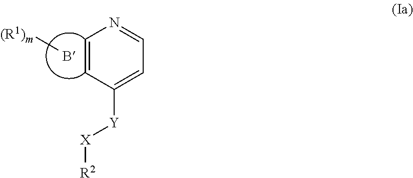

wherein
B′ is selected from a benzene, pyridine, pyrrole and pyrazole ring; and
R¹, R², X, Y and m are as defined above for formula (I).

In formulae (I), (I′) and (Ia), when m is 1 or 2 the group or groups R¹ may be present on any available ring position in the fused ring system. A group R¹ may thus be present on either or both of the ring B (or B′) and the pyridine ring.

In formulae (I), (I′) and (Ia), B (or B′) is most typically a benzene ring.

In formulae (I), (I′) and (Ia), when Y is a heteroaryl or dihydroheteroaryl ring it may be connected to the pyridine ring above and to the linker X below by either a ring carbon atom or a ring heteroatom. Typically it is connected to the pyridine ring via a ring carbon atom. Y is connected to the linker X by either a ring carbon atom or a ring heteroatom; thus it may be C-linked or N-linked. Typically, when Y is a heteroaryl or diheteroaryl ring which is connected to X via a nitrogen atom on Y, i.e. when Y is N-linked to X, X is not —O—, —S—, —NR³—, —NR³SO—, —NR³SO₂—, —N(SO₂R³)—, —NR³CO—, —NR³CONR³—, —NR³CS— or —NR³CSNR³—. When Y is N-linked to X, X is typically selected from —C(R³)₂—, —CO—, —CONR³—, —CSNR³—, —SO— and —SO₂—.

Y in formulae (I), (I′) and (Ia) is typically a ring selected from pyrazole, pyrimidine, thiazole, oxazole, pyrrole, dihydropyrazole, thiophene, indazole, furan and benzene. More typically it is a ring selected from pyrazole, pyrimidine, thiazole, oxazole, pyrrole, dihydropyrazole, thiophene, indazole and furan. Even more typically Y is a ring selected from pyrazole, thiazole, oxazole, pyrrole, dihydropyrazole, thiophene, indazole and furan.

In formulae (I), (I′) and (Ia), linker X is typically selected from —O—, —S—, —SO—, —SO₂—, —NR³SO—, —NR³SO₂—, —N(SO₂R³)—, —CO—, —CONR³—, —NR³CO—, —NR³CONR³—, —NR³CS— and —NR³CSNR³. More typically X is selected from —S—, —SO₂—, —CO—, —CONR³—, —NR³CO—, —NR³CONR³— and —N(SO₂R³)—. R³ in the definitions of linker X is typically H or C₁-C₆ alkyl.

In formulae (I), (I′) and (Ia) the terminal group R² is typically an aryl group, for instance a benzene ring.

When R² in formulae (I), (I′) and (Ia) is substituted it bears 1, 2 or 3 substituents selected from any of the options given above as suitable substituents for an aryl group. Typically it is substituted by an H-bond acceptor group and optionally further substituted by a C₁-C₆ alkyl group. Suitable examples of H-bond acceptor groups in this context include NO₂, F, —CN, —OR⁸, —CO₂R⁸, —SO₂R⁸, —SO₂NR⁸, —SOR⁸, —CONR⁸, —NR⁸COR⁸, —NR⁸CON(R⁸)₂, —NR⁸COOR⁸ and 5- or 6-membered heteroaryl groups, wherein R⁸ is as defined above. The substituent or substituents is or are more typically selected from NO₂, F, —CN, —OCH₃, —CO₂CH₃, oxadiazole and thiazole groups, optionally together with CH₃.

In formulae (I), (I′) and (Ia) the or each substituent R¹ is present on either the ring B (or B′) or the pyridine ring. The pyridine moiety may thus be either unsubstituted or substituted by one or two groups R¹. R¹ may occupy one or both of ring positions 2 and 3 on the pyridine ring. Typically the pyridine ring is unsubstituted or it is mono-substituted at position 2 or 3 by a group —O(C(R³)₂)ₙNR⁴R⁵, —NR³(C(R³)₂)ₙNR⁴R⁵ or —CONR⁴R⁵, in which R³, R⁴ and R⁵ are as defined above. For instance, the pyridine ring is unsubstituted or it is mono-substituted at position 2 or 3 by a group —O(CH₂)ₙNR⁴R⁵, —NR(CH₂)ₙNR⁴R⁵ in which R⁴ and R⁵ together form, with the nitrogen atom to which they are attached, a morpholine ring.

In a particular embodiment the fused pyridine of formula (I) or (I′) has the following formula (Ib):

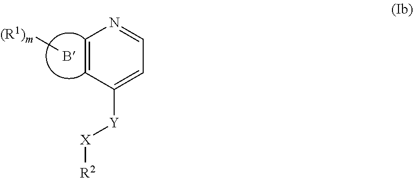

wherein:
B′ is a benzene ring;
each R¹, which are the same or different when m is greater than 1, is selected from H, halogen, CN, OR³, alkyl, alkenyl, alkynyl, CF₃, —O(C(R³)₂)ₙNR⁴R⁵, —NR³(C(R³)₂)ₙNR⁴R⁵, —NR⁴R⁵, —CONR⁴R⁵, —SO₂NR⁴R⁵, NO₂, —S(O)ₚR³, —CO₂R³, —NR³COR³, —NR³SO₂R³ and R⁶;
m is 0, 1 or 2;
Y is a pyrazole, dihydropyrazole, pyrimidine, thiazole or oxazole ring;
X is selected from —S—, —SO₂—, —NR³SO—, —NR³SO₂—, —N(SO₂R³)—, —CO—, —CONR³—, —NR³CO— and —NR³CONR³—;
R² is aryl which is unsubstituted or substituted;
R³ is selected from H and C₁-C₆ alkyl;
R⁴ and R⁵, which are the same or different, are each selected from H, C₁-C₆ alkyl, C₁-C₆ alkenyl and C₁-C₆ alkynyl, or R⁴ and R⁵ together with the N atom to which they are attached form a 5- or 6-membered heterocyclic ring containing 0, 1 or more additional heteroatoms selected from N, O and S;
n is 2 or 3;
p is 1 or 2;
R⁶ is an aryl or heteroaryl ring which is unsubstituted or substituted; and
R′ is H or C₁-C₆ alkyl.

Specific examples of fused pyridines of formulae (I) and (I′) include those listed in the following table:

| No. | Compound structure | Compound name |
|---|---|---|
| 1 | 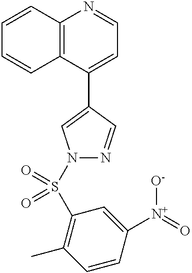 | 4-[1-(2-Methyl-5-nitro- benzenesulfonyl)-1H- pyrazol-4-yl]-quinoline |
| 2 | 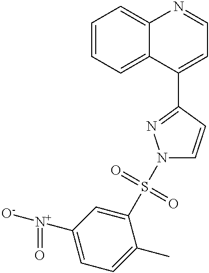 | 4-[1-(2-Methyl-5-nitro- benzenesulfonyl)-1H- pyrazol-3-yl]-quinoline |
| 3 | 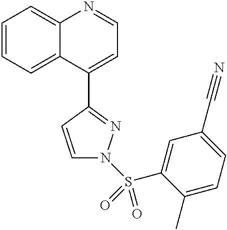 | 4-Methyl-3-(3-quinolin-4- yl-pyrazole-1-sulfonyl)- benzonitrile |
| 4 | 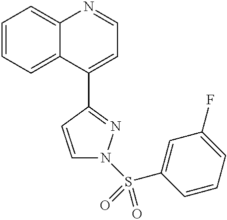 | 4-[1-(3-Fluoro- benzenesulfonyl)-1H- pyrazol-3-yl]-quinoline |
| 5 | 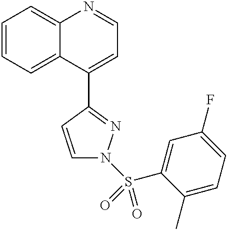 | 4-[1-(5-Fluoro-2-methyl- benzenesulfonyl)-1H- pyrazol-3-yl]-quinoline |
| 6 | 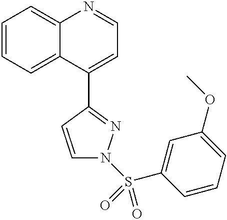 | 4-[1-(3-Methoxy- benzenesulfonyl)-1H- pyrazol-3-yl]-quinoline |
| 7 | 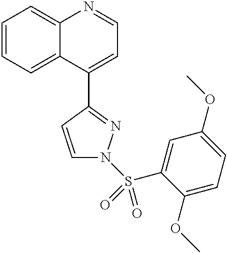 | 4-[1-(2,5-Dimethoxy- benzenesulfonyl)-1H- pyrazol-3-yl]-quinoline |
| 8 | 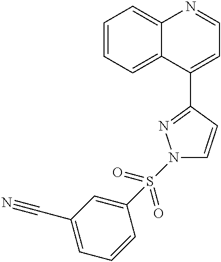 | .3-(3-Quinolin-4-yl- pyrazole-1-sulfonyl)- benzonitrile |
| 9 | 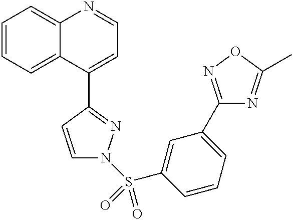 | 4-{1-[3-(5-Methyl- [1,2,4]oxadiazol-3-yl)- benzenesulfonyl]-1H- pyrazol-3-yl}-quinoline |
| 10 | 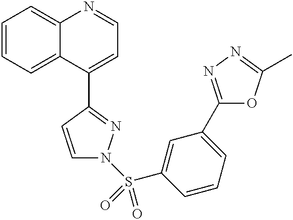 | 4-{1-[3-(5-Methyl- [1,3,4]oxadiazol-2-yl)- benzenesulfonyl]-1H- pyrazol-3-yl}-quinoline |
| 11 | 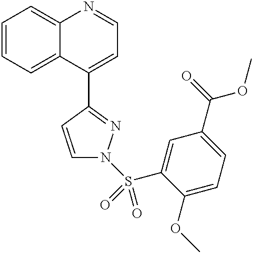 | Methyl 4-methoxy-3-(3- quinolin-4-yl-pyrazole-1- sulfonyl)-benzoate |
| 12 | 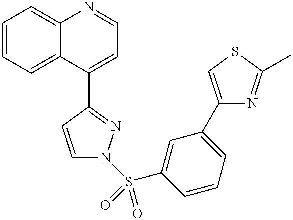 | 4-{1-[3-(2-Methyl-thiazol- 4-yl)-benzenesulfonyl]- 1H-pyrazol-3-yl}- quinoline |
| 13 | 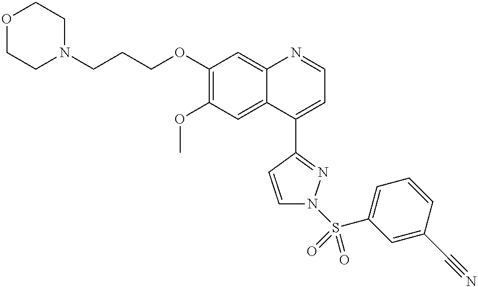 | 3-{3-[6-Methoxy-7-(3- morpholin-4-yl-propoxy)- quinolin-4-yl]-pyrazole-1- sulfonyl}-benzonitrile |
| 14 | 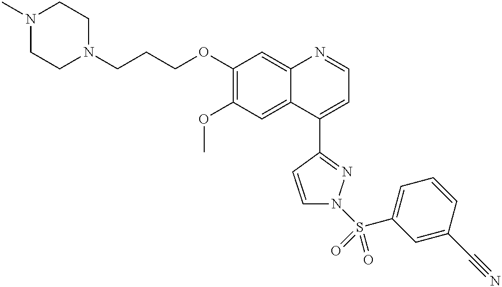 | 3-(3-{6-Methoxy-7-[3-(4- methyl-piperazin-1-yl)- propoxy]-quinolin-4-yl}- pyrazole-1-sulfonyl)- benzonitrile |
| 15 | 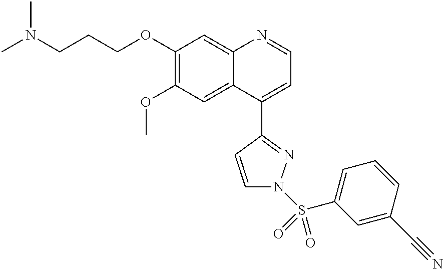 | 3-{3-[7-(3- Dimethylamino-propoxy)- 6-methoxy-quinolin-4-yl]- pyrazole-1-sulfonyl}- benzonitrile |
| 16 | 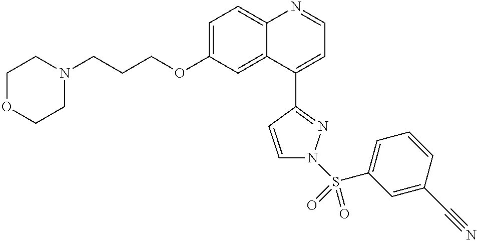 | 3-{3-[6-(3-Morpholin-4- yl-propoxy)-quinolin-4- yl]-pyrazole-1-sulfonyl}- benzonitrile |
| 17 | 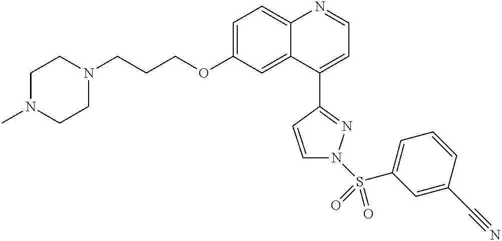 | 3-(3-{6-[3-(4-Methyl- piperazin-1-yl)-propoxy]- quinolin-4-yl}-pyrazole-1- sulfonyl)-benzonitrile |
| 18 | 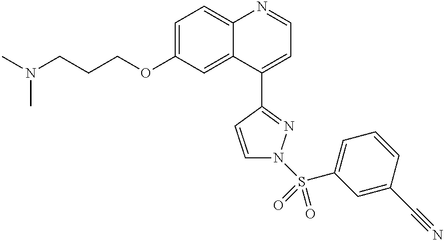 | 3-{3-[6-(3- Dimethylamino-propoxy)- quinolin-4-yl]-pyrazole-1- sulfonyl}-benzonitrile |
| 19 | 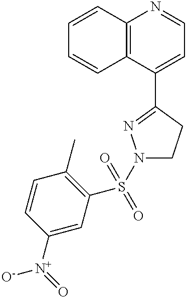 | 4-[1-(2-Methyl-5-nitro- benzenesulfonyl)-4,5- dihydro-1H-pyrazol-3-yl]- quinoline |
| 20 | 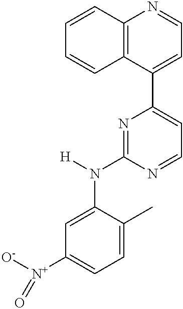 | (2-Methyl-5-nitro-phenyl)- (4-quinolin-4-yl- pyrimidin-2-yl)-amine |
| 21 | 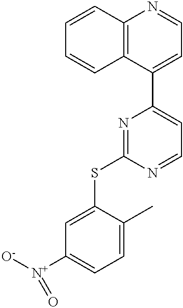 | 4-[2-(2-Methyl-5-nitro- phenylsulfanyl)-pyrimidin- 4-yl]-quinoline |
| 22 | 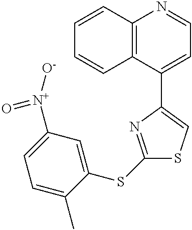 | 4-[2-(2-Methyl-5-nitro- phenylsulfanyl)-thiazol-4- yl]-quinoline |
| 23 | 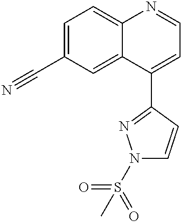 | 4-(1-Methanesulfonyl-1H- pyrazol-3-yl)-quinoline-6- carbonitrile |

and the pharmaceutically acceptable salts thereof.

A compound of the invention may be prepared according to the following scheme 1:

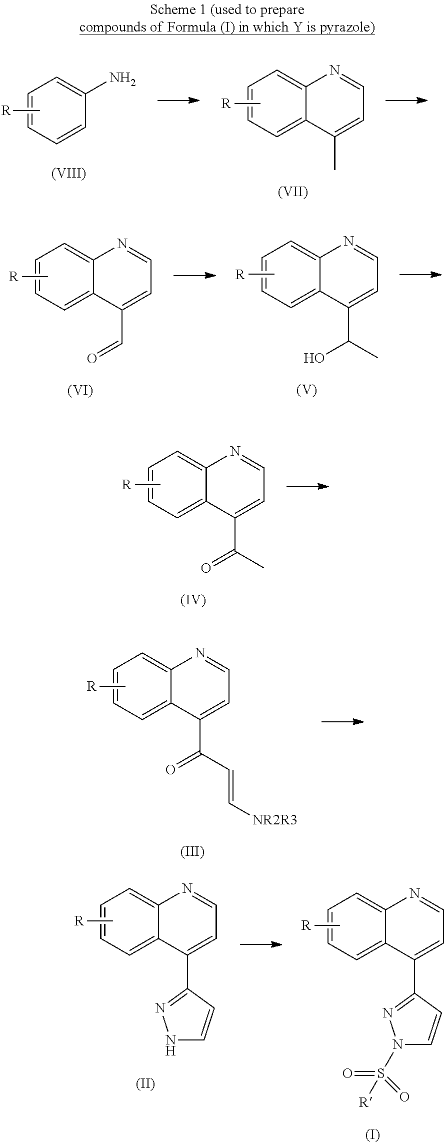

Hence, an appropriately substituted aniline of formula (VIII) may be converted into a substituted quinoline of formula (VII) using an oxidative cyclisation with methyl vinyl ketone. This reaction is a modified version of the Slump cyclisation and utilizes an iron (III) salt such as a FeCl₃ and an optional zinc salt, such as zinc chloride. A suitable solvent for this reaction is acetic acid, methanol or ethanol, more preferably ethanol. The reaction temperature is typically at the reflux temperature of the solvent.

A compound of formula (VI) may be prepared by treatment of a compound of formula (VII) with a suitable oxidizing agent. An example of a suitable oxidizing agent is selenium dioxide. A suitable solvent for this reaction is dioxane Treatment of the aldehyde of formula (VI) with an organometallic such as methyl lithium or methyl magnesium bromide gives a compound of formula (V). A suitable solvent for this reaction is an ether such as tetrahydrofuran. Oxidation of a compound of formula (V) using a suitable oxidizing agent provides the ketone of formula (IV). Many oxidizing conditions and reagents would be suitable for this reaction including Swern conditions, pyridinium chlorochromate, pyridinium dichromate, manganese dioxide, Dess-Martin conditions and N-methylmorpholine oxide with tetrapropylammonium perruthenate. More preferable is N-methylmorpholine oxide with tetrapropylammonium perruthenate.

A compound of formula (III) may be prepared by treatment of a compound of formula (IV) with a reagent of formula (MeO)₂CHNR2R3, wherein R2 and R3 are as described previously, such as N,N-dimethylformamide dimethylacetal. This reaction is preferably carried out at an elevated temperature with no additional solvent. Compounds of formula (III) may exist with either E or Z geometry, more commonly with E geometry.

A compound of formula (II) may be prepared by treatment of a compound of formula (III) with hydrazine in a suitable solvent. A suitable solvent may be an alcohol such as ethanol and the reaction is usually carried out at an elevated temperature.

A compound of formula (I) may be prepared by reaction of a compound of formula (II) with a compound of formula R′SO₂Cl. A suitable solvent for this reaction could be pyridine, dichloromethane, chloroform or acetonitrile. A suitable base for this reaction could be pyridine, triethylamine or potassium carbonate.

Compounds of formula 1 may also be prepared by Scheme 2

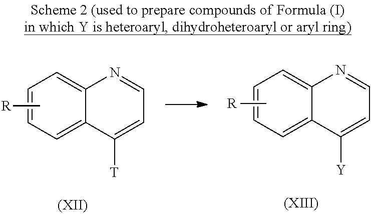

Hence compounds of formula (XII), wherein T is selected from I, Br, Cl or OTf, may be converted into compounds of formula (XIII), wherein Y is as described above, by treatment of a group Y—B(OH)₂, or an ester thereof, using Suzuki cross coupling conditions. Conventional Suzuki conditions may be used for this step. Thermal of microwave heating may be employed and a palladium catalyst such as PdCl₂(PPh₃)₂ or Pd(PPh₃)₄ is used. Compounds of formula (XIII) may be converted into compounds of formula (I) using methods described in the other schemes herein.

By analogy, compounds of formula (XIV) may be made by using Suzuki conditions with a compound of formula X—Y—B(OH)₂, or an ester thereof. Compounds of formula (XIV) may be converted into compounds of formula (I) using methods described in the other schemes herein.

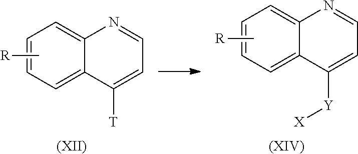

By analogy, compounds of formula (I) may be made by using Suzuki conditions with a compound of formula R′—X—Y—B(OH)₂, or an ester thereof.

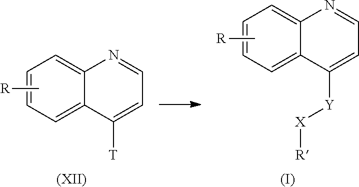

For the preparation of compounds of formula (I) in which the heterocycle is a pyrimidine, Schemes 3 and 4 may be used.

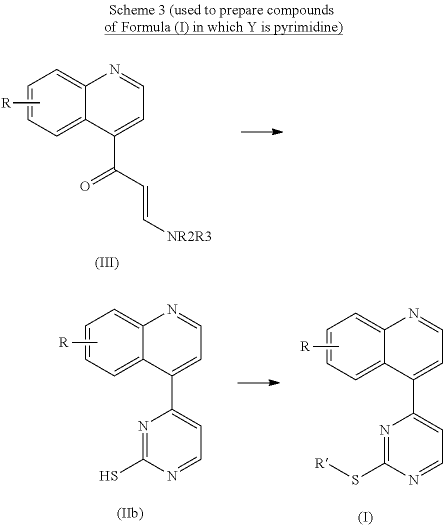

Hence, a compound of formula (III), prepared as described above, was cyclised to a compound of formula (IIb) using thiourea in the presence of a base in a suitable solvent. A suitable solvent is an alcohol such as ethanol and the reaction is typically carried out at an elevated temperature. A typical base for this reaction is potassium hydroxide. Compounds of formula (I) are prepared by treatment of a compound of formula (IIb) with a compound of formula R′X where X is selected from halogen, OTf and N₂⁺X⁻. Typically compounds of Formula (I) are prepared by treatment with compounds of formula R′N₂⁺BF₄⁻ in a suitable aprotic solvent such as DMSO in the presence of a base such a sodium hydride.

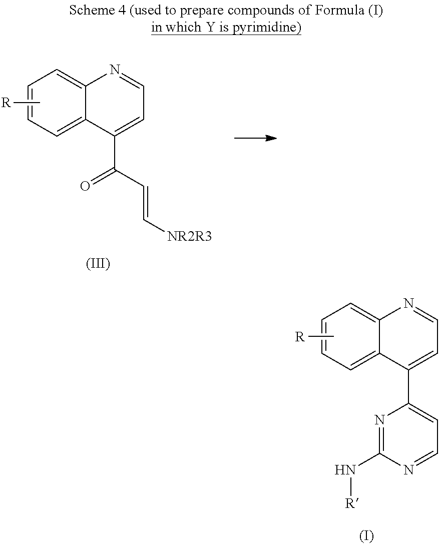

Hence a compound of formula (I), in which the heterocyclic ring is a pyrimidine may be prepared by treatment of a compound of formula (III), prepared as described above, with a compound of formula R′NHC(NH)NH2 in a suitable solvent. This reaction is typically performed at an elevated temperature in the presence of base.

For the preparation of compounds of formula (I) in which the heterocycle is a pyrazoline, Scheme 5 may be used.

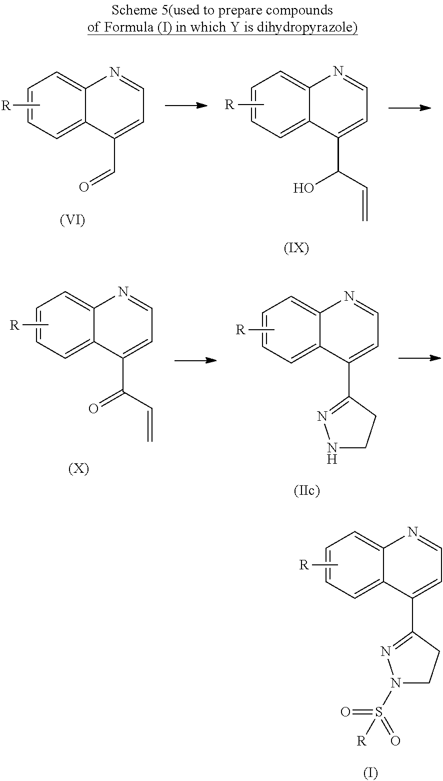

Hence a compound of formula (VI), as described above, may be converted to a compound of formula (IX) by treatment with vinyl magnesium bromide, in a suitable solvent such as tetrahydrofuran. Oxidation of a compound of formula (IX) using a suitable oxidizing agent provides the ketone of formula (X). Many oxidizing reagents would be suitable for this reaction including Swern conditions, pyridinium chlorochromate, pyridinium dichromate, manganese dioxide, Dess-Martin conditions and N-methylmorpholine oxide with tetrapropylammonium perruthenate. More preferable is N-methylmorpholine oxide with tetrapropylammonium perruthenate. A compound of formula (IIc) may be prepared by treatment of a compound of formula (X) with hydrazine in a suitable solvent. A suitable solvent may be an alcohol such as ethanol and the reaction is usually carried out at an elevated temperature.

A compound of formula (I) may be prepared by reaction of a compound of formula (IIc) with a compound of formula R′SO₂Cl. A suitable solvent for this reaction could be pyridine, dichloromethane, chloroform or acetonitrile. A suitable base for this reaction could be pyridine, triethylamine or potassium carbonate.

For the preparation of compounds of formula (I) in which the heterocycle is a thiazole, Scheme 6 may be used.

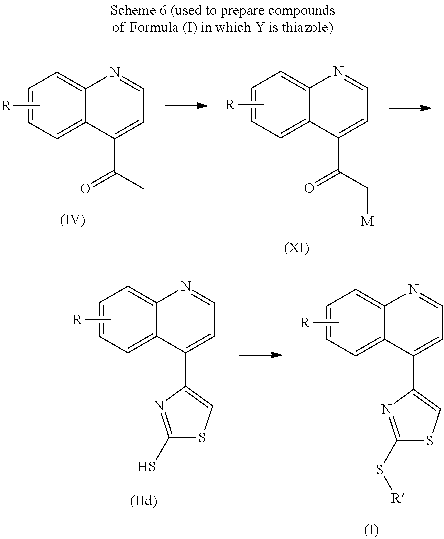

Hence a compound of formula (IV) is halogenated to form a compound of formula (XI) wherein M is selected from F, Cl, Br, and I. Typically M is Cl or Br, more typically Br.

Treatment of a compound of formula (XI) with ammonium dithiocarbamate provides a compound of formula (IId). Compounds of formula (I) are prepared by treatment of a compound of formula (IId) with a compound of formula R′X where X is selected from halogen, OTf and N₂⁺X⁻. Typically compounds of Formula (I) are prepared by treatment with compounds of formula R′N₂⁺BF₄⁻ in a suitable aprotic solvent such as DMSO in the presence of a base such a sodium hydride

A fused pyridine of formula (I) or (I′) may be converted into a pharmaceutically acceptable salt, and a salts may be converted into the free compound, by conventional methods. Examples of pharmaceutically acceptable salts include acid addition salts with inorganic acids such as hydrochloric acid, hydrobromic acid, hydroidic acid, sulphuric acid, nitric acid and phosphoric acid; and organic acids such as formic acid, acetic acid, trifluoroacetic acid, propionic acid, oxalic acid, malonic acid, succinic acid, fumaric acid, maleic acid, lactic acid, malic acid, tartaric acid, citric acid, methanesulfonic acid, ethanesulfonic acid, aspartic acid and glutamic acid. In the case of compounds of the invention bearing a carboxylic acid substituent, the salts include the salts of alkali and alkaline earth metals and ammonium, for instance the salts of sodium, potassium, magnesium, calcium and ammonium. The latter are prepared by treating the free quinoline of formula (I), or an acid addition salt thereof, with the corresponding metal base or ammonia.

The fused pyridines of formula (I) or (I′) and their salts may exist as hydrates or solvates.

Compounds of the present invention have been found in biological tests to be inhibitors of c-Met. A compound of the present invention may thus be used as an inhibitor of c-Met. Accordingly, a compound of the present invention can be used to treat or ameliorate cancer or to prevent the metastasis of cancer. It can also be used to treat an immunological disorder, a cardiovascular disorder or an ocular disorder.

In one embodiment, a human patient is treated with a compound of the invention as defined above and a pharmaceutically acceptable carrier, adjuvant, or vehicle, wherein said compound of the invention is present in an amount to inhibit c-Met activity.

Cancers in which Met expression has been demonstrated in tumour biopsies, and which may therefore be treated with a compound of the invention, include bladder carcinoma, breast, cervical, colorectal, oesophageal, gastric, head and neck, kidney, liver, lung, nasopharyngeal, ovarian, pancreatic, prostate, thyroid, osteosarcoma, synovial sarcoma, rhabdomyosarcoma, malignant fibrous histiocytoma, leiomyosarcoma, Kaposi's sarcoma, multiple myeloma, lymphomas, adult T-call leukemia, glioblastomas/astroyomas, melanoma, mesotheklioma, and Wilms' tumour. The metastasis of these cancers may also be prevented with a compound of the invention.

Cardiovascular diseases which can be treated according to the methods of this invention include, but are not limited to atherosclerosis, stroke and myocardial infarction.

Immunological disorders which can be treated according to the invention include rheumatoid arthritis, psoriasis, diabetes, multiple sclerosis and GVHD (graft versus host disease).

Ocular disorders which can be treated according to the invention include macular degeneration and diabetic retinopathy.

A compound of the present invention can be administered in a variety of dosage forms, for example orally such as in the form of tablets, capsules, sugar- or film-coated tablets, liquid solutions or suspensions or parenterally, for example intramuscularly, intravenously or subcutaneously. The compound may therefore be given by injection or infusion.

The dosage depends on a variety of factors including the age, weight and condition of the patient and the route of administration. Daily dosages can vary within wide limits and will be adjusted to the individual requirements in each particular case. Typically, however, the dosage adopted for each route of administration when a compound is administered alone to adult humans is 0.0001 to 50 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

Typically a dose to treat human patients may range from about 10 mg to about 1000 mg of a compound of the invention. A typical dose may be about 100 mg to about 300 mg of the compound. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

A compound is formulated for use as a pharmaceutical or veterinary composition also comprising a pharmaceutically or veterinarily acceptable carrier or diluent. The compositions are typically prepared following conventional methods and are administered in a pharmaceutically or veterinarily suitable form. The compound may be administered in any conventional form, for instance as follows:

A) Orally, for example, as tablets, coated tablets, dragees, troches, lozenges, aqueous or oily suspensions, liquid solutions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, dextrose, saccharose, cellulose, corn starch, potato starch, calcium phosphate or sodium phosphate; granulating and disintegrating agents, for example, maize starch, alginic acid, alginates or sodium starch glycolate; binding agents, for example starch, gelatin or acacia; lubricating agents, for example silica, magnesium or calcium stearate, stearic acid or talc; effervescing mixtures; dyestuffs, sweeteners, wetting agents such as lecithin, polysorbates or lauryl sulphate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Such preparations may be manufactured in a known manner, for example by means of mixing, granulating, tableting, sugar coating or film coating processes.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides for example polyoxyethylene sorbitan monooleate.

The said aqueous suspensions may also contain one or more preservatives, for example, ethyl or n-propyl p-hydroxybenzoate, one or more colouring agents, such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by this addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occuring phosphatides, for example soy bean lecithin, and esters or partial esters derived from fatty acids an hexitol anhydrides, for example sorbitan mono-oleate, and condensation products of the said partial esters with ethylene oxide, for example polyoxyethylene sorbitan monooleate. The emulsion may also contain sweetening and flavouring agents. Syrups and elixirs may be formulated with sweetening agents, for example glycerol, sorbitol or sucrose. In particular a syrup for diabetic patients can contain as carriers only products, for example sorbitol, which do not metabolise to glucose or which only metabolise a very small amount to glucose.

Such formulations may also contain a demulcent, a preservative and flavouring and coloring agents;

B) Parenterally, either subcutaneously, or intravenously, or intramuscularly, or intrasternally, or by infusion techniques, in the form of sterile injectable aqueous or oleaginous suspensions. This suspension may be formulated according to the known art using those suitable dispersing of wetting agents and suspending agents which have been mentioned above. The sterile injectable preparation may also be a sterile injectable solution or suspension in a non-toxic paternally-acceptable diluent or solvent, for example as a solution in 1,3-butane diol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables;

C) By inhalation, in the form of aerosols or solutions for nebulizers;

D) Rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols;

E) Topically, in the form of creams, ointments, jellies, collyriums, solutions or suspensions.

F) Vaginally, in the form of pessaries, tampons, creams, gels, pastes, foams or spray formulations containing in addition to the active ingredient such carriers as are known in the art to be appropriate.

The invention will be further illustrated in the following Examples

### General Experimental Details:

### NMR Spectroscopy

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz. Shifts are given in ppm relative to tetramethylsilane.

### Purification by Column Chromatography

Compounds purified by column chromatography were purified using silica gel or Isolute® cartridge or Redisep® cartridge, eluting with gradients from 100-0 to 0-100% of cyclohexane/EtOAc, or from 100-0 to 0-100% pentane/EtOAc or from 100-0 to 70-30% DCM/MeOH (with or without the addition of NH₃ 0.1%). ‘Silica gel’ refers to silica gel for chromatography, 0.035 to 0.070 mm (220 to 440 mesh) (e.g. Fluka silica gel 60), and an applied pressure of nitrogen up to 10 p.s.i accelerated column elution. Where thin layer chromatography (TLC) has been used, it refers to silica gel TLC using plates, typically 3×6 cm silica gel on aluminium foil plates with a fluorescent indicator (254 nm), (e.g. Fluka 60778).

### Purification by Preparative HPLC:

Compounds purified by preparative HPLC were purified using a C18-reverse-phase column (100×22.5 mm i.d Genesis column with 7 μm particle size, UV detection at 230 or 254 nm, flow 5-15 mL/min), or a Phenyl-Hexyl column (250×21.2 mm i.d. Gemini column with 5 μM particle size, UV detection at 230 or 254 nm, flow 5-20 mL/min), eluting with gradients from 100-0% to 0-100% water/acetonitrile or water/MeOH containing 0.1% TFA or water/acetonitrile containing 0.1% formic acid. The free base was liberated by partitioning between EtOAc and a sat. solution of sodium bicarbonate. The organic layer was dried (MgSO₄) and concentrated in vacuo. Alternatively, the free base was liberated by passing through an Isolute® SCX-2 cartridge, eluting with NH₃ in methanol.

Abbreviations used in the experimental section:
aq.=aqueous
BOC=t-Butoxycarbonyl
bs=broad singlet (NMR)
Cs₂CO₃=cesium carbonate
d=doublet (NMR)
DCM=dichloromethane
DIPEA=diisopropylethylamine
DMA=dimethylacetamide
DMAP=dimethylaminopyridine
DMF=dimethylformamide
DMSO=dimethylsulfoxide
eq.=equivalents
EtOAc=ethyl acetate
EtOH=ethanol
h=hour(s)
HATU=O-(7-Azabenzotriazol-1-yl)-N,N,N′,N′-tetramethyluronium hexafluorophosphate
HCl=hydrochloric acid
H₂O=water
HPLC=high pressure liquid chromatography
IMS=industrial methylated spirit
iPrOH=isopropanol
LCMS=liquid chromatography mass spectrometry
M=molar
m=multiplet (NMR)
MeOH=methanol
mg=milligram
MgSO₄=magnesium sulphate
min=minute(s)
mL=millilitre
Na₂CO₃=sodium carbonate
NaHCO₃=sodium hydrogen carbonate
NaOH=sodium hydroxide
Na₂SO₄=sodium sulfate
NMR=nuclear magnetic resonance
q=quartet (NMR)
Rt=retention time
RT=room temperature
sat=saturated
t=triplet (NMR)
TFA=trifluoroacetic acid
THF=tetrahydrofuran
TLC=thin layer chromatography

### Reference Example 1

### 4-Formyl-quinoline-6-carbonitrile

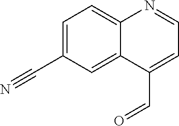

To a mixture of 4-aminobenzonitrile hydrochloride (2 g), ZnCl₂ (288 mg), FeCl₃ (4.39 g) and ethanol (50 mL) at 65° C. was added methyl vinyl ketone (1.69 mL). The reaction mixture was heated to reflux for 24 hours and then cooled. The reaction mixture was diluted with water, basified (sodium carbonate) and then extracted into chloroform. The solvent was removed in vacuo, and the residue was purified using flash chromatography to yield 4-methyl-quinoline-6-carbonitrile (1.56 g) A mixture of 4-methyl-quinoline-6-carbonitrile (0.8 g.), selenium dioxide (528 mg) in dioxane (10 mL) and water (1 mL) was heated together at 90° C. overnight. The mixture was then diluted with CH₂Cl₂ then filtered. The filtrate was washed with aqueous sodium carbonate solution, dried (MgSO₄) concentrated in vacuo and the product isolated by flash chromatography to yield 4-formyl-quinoline-6-carbonitrile (510 mg).

The following compounds were prepared in a similar manner starting from the appropriate aniline.
7-Benzyloxy-6-methoxy-quinoline-4-carbaldehyde was prepared from 3-benzyloxy-4-methoxyaniline;
6-Benzyloxy-quinoline-4-carbaldehyde was prepared from 4-benzyloxyaniline;

### Reference Example 2

### 4-Acetyl-quinoline-6-carbonitrile

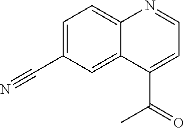

To a solution of 4-formyl-quinoline-6-carbonitrile (200 mg) in THF (5 mL) at −40° C. was added dropwise methylmagnesium bromide (0.36 mL of a 3 M solution in diethyl ether). The reaction mixture was allowed to warm to room temperature over 3 h and was then quenched with brine (20 mL). The mixture was extracted into ethyl acetate (2×20 mL) and the combined organics washed with water (20 mL), dried (MgSO₄), reduced in vacuo and purified using column chromatography to give 4-(1-hydroxy-ethyl)-quinoline-6-carbonitrile.

To a solution of 4-(1-hydroxy-ethyl)-quinoline-6-carbonitrile (450 mg) and N-methylmorpholine oxide (798 mg) in dichloromethane (10 mL) was added molecular sieves. After stirring at room temperature for 10 min, tetrapropylammonium perruthenate (80 mg) was added and the reaction mixture was stirred at room temperature for 1 h. The mixture was then filtered through Celite and the filtrate reduced in vacuo and purified by column chromatography to give 4-acetyl-quinoline-6-carbonitrile as an off-white solid (263 mg).

The following compounds were prepared in a similar manner starting from the appropriate aldehyde.
1-Quinolin-4-yl-ethanone was prepared from quinoline-4-carboxaldehyde;
1-(7-Benzyloxy-6-methoxy-quinolin-4-yl)ethanone was prepared from 7-benzyloxy-6-methoxy-quinoline-4-carbaldehyde;
1-(6-Benzyloxy-quinolin-4-yl)-ethanone was prepared from 6-benzyloxy-quinoline-4-carbaldehyde

### Reference Example 3

### 1-[7-(3-Chloro-propoxy)-6-methoxy-quinolin-4-yl]-ethanone

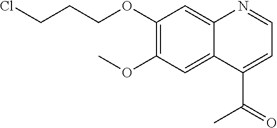

A stirred solution of 1-(7-benzyloxy-6-methoxy-quinolin-4-yl)-ethanone (2.30 g; 7.4 mmol) and methanesulfonic acid (2 ml; 31 mmol) in trifluoroacetic acid (40 ml) was heated at reflux temperature for 3 h. The reaction mixture was cooled to r.t., volatiles removed in vacuo and the residue was basified with saturated Na₂CO₃ solution. The resulting solid was collected by filtration and washed with MeOH then Et₂O and dried to give 1-(7-hydroxy-6-methoxy-quinolin-4-yl)-ethanone as an off-white solid (1.40 g; 87%).

To a stirred mixture of 1-(7-hydroxy-6-methoxy-quinolin-4-yl)-ethanone (1.40 g; 6.44 mmol) and K₂CO₃ (4.45 g; 32.2 mmol) in anhydrous DMF was added 1-bromo-3-chloropropane (3.0 ml; 30.3 mmol) and the reaction mixture was stirred at r.t. for 5 h upon which time it was diluted with brine (40 ml) and extracted with CH₂Cl₂ (3×40 ml). The combined organic layers were dried (Na₂SO₄), concentrated and purified by flash chromatography to give 1-[7-(3-chloro-propoxy)-6-methoxy-quinolin-4-yl]-ethanone as a tan solid (1.35 g; 71%). 1-[6-(3-Chloro-propoxy)-quinolin-4-yl]-ethanone was prepared in a similar manner using 1-(6-benzyloxy-quinolin-4-yl)-ethanone.

### Reference Example 4

### 6-Methoxy-7-(3-morpholin-4-yl-propoxy)-4-(1H-pyrazol-3-yl)-quinoline

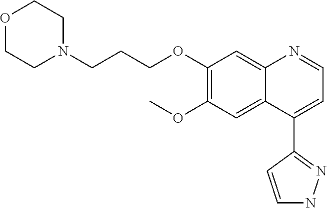

A stirred solution of 1-[7-(3-chloro-propoxy)-6-methoxy-quinolin-4-yl]-ethanone (0.50 g; 1.7 mmol), and N,N-diisopropylethylamine (0.5 ml; 2.9 mmol) in morpholine was heated in a sealed Reactivial at 100° C. for 2 d. The reaction mixture was cooled to r.t., diluted with brine (30 ml) and extracted with CH₂Cl₂ (2×40 ml). The combined organic layers were dried (Na₂SO₄) and concentrated to an orange gum. This gum was dissolved in N,N-dimethylformamide dimethylacetal (5 ml) and heated at reflux temperature for 4 h. Volatiles were removed in vacuo, the dark brown residue was dissolved in EtOH (5 ml) the solution was treated with hydrazine monohydrate (0.5 ml; 10 mmol) and heated at relux temperature for 1 h. The reaction mixture was cooled, diluted with water (40 ml) and the resulting solid was collected by filtration and dried to give 6-methoxy-7-(3-morpholin-4-yl-propoxy)-4-(1H-pyrazol-3-yl)-quinoline as a tan solid (0.52 g; 84%).

The following compounds were made in a similar manner from the appropriate starting materials;
6-Methoxy-7-[3-(4-methyl-piperazin-1-yl)-propoxy]-4-(1H-pyrazol-3-yl)-quinoline was prepared from N-methylpiperazine and 1-[7-(3-chloro-propoxy)-6-methoxy-quinolin-4-yl]-ethanone;
3-[6-Methoxy-4-(1H-pyrazol-3-yl)-quinolin-7-yloxy]-propyl}-dimethyl-amine was prepared from dimethylamine and 1-[7-(3-chloro-propoxy)-6-methoxy-quinolin-4-yl]-ethanone;
6-(3-Morpholin-4-yl-propoxy)-4-(1H-pyrazol-3-yl)-quinoline was prepared from 1-[6-(3-chloro-propoxy)-quinolin-4-yl]-ethanone and morpholine;
6-[3-(4-Methyl-piperazin-1-yl)-propoxy]-4-(1H-pyrazol-3-yl)-quinoline was prepared from 1-[6-(3-chloro-propoxy)-quinolin-4-yl]-ethanone and N-methylpiperazine;
Dimethyl-{3-[4-(1H-pyrazol-3-yl)-quinolin-6-yloxy]-propyl}-amine was prepared from 1-[6-(3-chloro-propoxy)-quinolin-4-yl]-ethanone and dimethylamine;

### Reference Example 5

### 4-(1H-Pyrazol-3-yl)-quinoline-6-carbonitrile

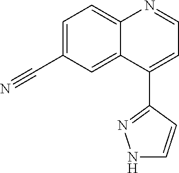

A mixture of 4-acetyl-quinoline-6-carbonitrile (260 mg) and N,N-dimethylformamide-dimethylacetetal (2 mL) was stirred at reflux for 2 h. After cooling to room temperature the solvent was removed in vacuo to give 4-((E)-3-dimethylamino-acryloyl)-quinoline-6-carbonitrile.

A mixture of 4-((E)-3-dimethylamino-acryloyl)-quinoline-6-carbonitrile (331 mg) and hydrazine monohydrate (0.08 mL) in ethanol (7 mL) was stirred at reflux for 4 h. After cooling to room temperature, the solid was filtered and washed with ethanol to give 4-(1H-pyrazol-3-yl)-quinoline-6-carbonitrile (190 mg).

The following compounds were made in a similar manner from the appropriate starting material.
4-(1H-Pyrazol-3-yl)-quinoline;

### Reference Example 6

### N-(2-Methyl-5-nitro-phenyl)-guanidine nitric acid salt

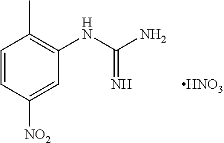

To a solution of 2-methyl-5-nitroaniline (1.90 g) in ethanol (3 mL) at 0° C. was added dropwise nitric acid (0.90 mL of a 70% solution in water). After complete addition, a solution of cyanamide (1.57 g) in water (1 mL) was added and the mixture was stirred at reflux for 4 h. After cooling to room temperature, the mixture was poured into diethyl ether (20 mL) and the resulting solid was filtered to give N-(2-methyl-5-nitro-phenyl)-guanidine nitric acid salt as a yellow solid (1.84 g).

### Reference Example 7

### 4-Quinolin-4-yl-pyrimidine-2-thiol

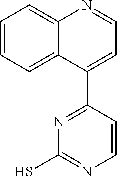

To a solution of 3-dimethylamino-1-quinolin-4-yl-propenone (992 mg) (see Reference Example 5) in ethanol (10 mL) was added 1 M potassium hydroxide in ethanol (4.39 mL) and thiourea (668 mg) and the reaction mixture was stirred at reflux for 4 h. After cooling to room temperature, the resulting solid was filtered and washed with diethyl ether to give 4-quinolin-4-yl-pyrimidine-2-thiol as a yellow solid (1.0 g).

### Reference Example 8

### 2-Methyl-5-nitro-benzenediazonium tetrafluoroborate

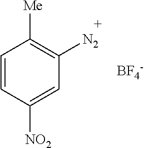

To a suspension of 2-methyl-5-nitroaniline (4.00 g) in 6 M aqueous HCl solution (40 mL) at 0° C. was added dropwise a solution of sodium nitrite (1.86 g) in water (10 mL) and the reaction mixture was stirred at 0° C. for 30 min. A solution of sodium tetrafluoroborate (4.00 g) in water (20 mL) was then added and the mixture stirred for a further 2 h. The solid was then filtered and washed with water to give 2-methyl-5-nitro-benzenediazonium tetrafluoroborate as a white solid (2.51 g).

### Reference Example 9

### 4-Quinolin-4-yl-thiazole-2-thiol

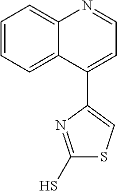

Gaseous ammonia (3.9 g) was passed into ethanol (25 mL) at 0° C. To this solution was added a pre-cooled mixture of carbon disulphide (7.6 g) and diethyl ether (20 mL). The reaction mixture was left at 0° C. for 2 h and then at room temperature for 72 h and was then filtered and the solid washed with diethyl ether to give ammonium dithiocarbamate (5.0 g).

To a solution of 1-quinolin-4-yl-ethanone (600 mg) (see Reference Example 2) in a 33% solution of hydrogen bromide in acetic acid (4 mL) at 0° C. was added dropwise bromine (533 mg). The reaction mixture was heated at 45° C. for 90 min and then at 75° C. for 1 h. After cooling to room temperature, the mixture was poured into diethyl ether (20 mL) and the solid was filtered and washed with diethyl ether to give 2-bromo-1-quinolin-4-yl-ethanone (800 mg).

To a suspension of 2-bromo-1-quinolin-4-yl-ethanone (800 mg) in ethanol (10 mL) was added ammonium dithiocarbamate (387 mg) and the mixture was stirred at room temperature for 16 h. The solid was then filtered and washed with diethyl ether and methanol and then redissolved in acetic acid (3 mL) and stirred at reflux for 2 h. After cooling to room temperature, the solid was filtered and washed with methanol to give 4-quinolin-4-yl-thiazole-2-thiol (258 mg).

### Reference Example 10

### 4-(4,5-Dihydro-1H-pyrazol-3-yl)-quinoline

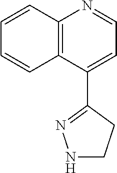

To a solution of 4-quinoline carboxaldehyde (750 mg) in THF (15 mL) at −20° C. was added vinylmagnesium bromide (5.73 mL of a 1 M solution in THF). The reaction mixture was warmed to room temperature over 3 h and then quenched with brine (20 mL). The mixture was then extracted into ethyl acetate (2×20 mL) and the combined organics were washed with brine (20 mL), dried (MgSO₄) and reduced in vacuo to give 1-quinolin-4-yl-prop-2-en-1-ol (880 mg).

To a solution of 1-quinolin-4-yl-prop-2-en-1-ol (880 g) and N-methyhnorpholine oxide (1.67 g) in dichloromethane (20 mL) was added molecular sieves. After stirring at room temperature for 10 min, tetrapropylammonium perruthenate (167 mg) was added and the reaction mixture was stirred at room temperature for 1 h. The mixture was then filtered through Celite and the filtrate reduced in vacuo and purified by column chromatography to give 1-quinolin-4-yl-propenone as a yellow oil (660 mg).

A mixture of 1-quinolin-4-yl-propenone (290 mg) and hydrazine monohydrate (0.09 mL) in ethanol (8 mL) was stirred at reflux for 4 h. After cooling to room temperature, the solvent was removed in vacuo to give 4-(4,5-dihydro-1H-pyrazol-3-yl)-quinoline.

### Reference Example 11

### 5-Cyano-2-methyl-benzenesulfonyl chloride

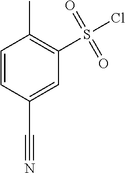

A mixture of 4-methyl-3-nitrobenzontitrile (2.41 g), 10% Pd on C (˜1 g) and ethanol was stirred under hydrogen. After 24 hours the reaction mixture was filtered through Celite, and the solvent reduced in vacuo and purified using flash chromatography to yield 3-amino-4-methyl-benzonitrile (1.21 g).

A solution of 3-amino-4-methyl-benzonitrile (454 mg) in conc. HCl (3.5 mL) and AcOH (4mL) was cooled to −10° C. To this was added a solution of NaNO₂ (284 mg) in water (2.1 ml) dropwise. After stirring for 15 minutes the mixture was added to a cooled (5° C.) solution of CuCl (85 mg) in AcOH (7 mL) that had previously been saturated with SO₂ gas. The reaction mixture was warmed to room temperature, SO₂ was removed in vacuo and then water was added to yield the title compound as a white solid which was collected by filtration. (520 mg).

### Example 1

### Compound Synthesis

### 4-[1-(2-Methyl-5-nitro-benzenesulfonyl)-1H-pyrazol-4-yl]-quinoline (1)

A mixture of 4-chloroquinoline (0.83 eq.), 4-(4,4,5,5-tetrethyl-[1,3,2]dioxaborolan-2-yl)-1H-pyrazole (1 eq.), Na₂CO₃ (aqueous, 3eq.) and PdCl₂(PPh₃)₂ (0.1 eq.) was heated at 130° C. in toluene (4 mL) and ethanol (2 mL) overnight. Aqueous work-up and flash chromatography gave 4-(1H-pyrazol-4-yl)-quinoline.

A solution of 4-(1H-pyrazol-4-yl)-quinoline (1 eq.), triethylamine (1.06 eq.) and 2-methyl-5-nitrobenzenesulfonyl chloride (1.49 eq.) in dry CH₂Cl₂ (5 mL) was stirred overnight at R.T. The mixture was concentrated in vacuo and product isolated by flash chromatography (33%).

NMR: (CDCl₃): 2.85 (s, 3H, CH3), 7.36 (d, H, ArH, J=4.42 Hz), 7.58-7.62 (m, 2H, 2× ArH), 7.78 (t, H, ArH, J=7.01 Hz), 7.98 (d, H, ArH, J=8.34 Hz), 8.05 (s, H, ArH), 8.18 (d, H, ArH, J=8.39 Hz), 8.42 (d, H, ArH, J=8.39 Hz), 8.51 (s, H, ArH), 8.92 (d, H, ArH, J=4.42 Hz), 9.01 (s, H, ArH)

MS: (ESI+): MH+=395.11

### 4-[1-(2-Methyl-5-nitro-benzenesulfonyl)-1H-pyrazol-3-yl]-quinoline (2)

To a mixture of 4-(1H-pyrazol-3-yl)-quinoline (240 mg) and potassium carbonate (184 mg) in acetonitrile (10 mL) was added 2-methyl-5-nitrobenzene sulfonyl chloride (266 mg). The reaction mixture was stirred at reflux for 18 h and then allowed to cool to room temperature. The reaction was partitioned between water (20 mL) and chloroform (20 mL) and the organic phase washed with brine (20 mL), dried (MgSO₄), reduced in vacuo and purified by column chromatography to give the title compound.

NMR: DMSO: 2.87 (3 H, s, Me), 6.91 (1H, d, J 2.85, Ar), 7.54-7.59 (3H, m, Ar), 7.74-7.8 (1H, m, Ar), 8.17 (1H, d, J 8.47, Ar), 8.37-8.39 (3H, m, Ar), 8.96 (1H, d, J 4.44, Ar) and 9.01 (1H, d, J 2.34, Ar).

MS: (ESI+): MH+ 395.11

The following compounds were made in a similar manner, using either pyridine or dichloromethane with triethylamine, or potassium carbonate in acetonitrile.

### 4-Methyl-3(3-quinolin-4-yl-pyrazole-1-sulfonyl)-benzonitrile (3)

Prepared from 4-(1H-pyrazol-3-yl)-quinoline and 5-cyano-2-methyl-benzenesulfonylchloride using conditions described above.

¹H NMR (CDCl₃): 2.81 (3H, s), 6.88 (1H, d, J 2.6), 7.47-7.59 (3H, m), 7.73-7.82 (2H, m), 8.16 (1H, d, J 8.2), 8.35-8.38 (2H, m), 8.96 (1H, d, J 4.5).

MS(ESI+): MH+ 375.

### 4-[1-(3-Fluoro-benzenesulfonyl)-1H-pyrazol-3-yl]-quinoline (4)

Prepared from 4-(1H-pyrazol-3-yl)-quinoline and 3-fluorobenzenesulfonylchloride using conditions described above.

¹H NMR (CDCl₃): 6.83 (1H, d, J 2.8), 7.33-7.42 (1H, m), 7.54-7.63 (3H, m), 7.73-7.78 (1H, m), 7.82-7.85 (1H, m), 7.93 (1H, d, J 8.0), 8.16 (1H, d, J 8.3), 8.29 (1H, d, J 2.8), 8.40 (1H, d, J 8.0), 8.96 (1H, d, J 4.4).

MS(ESI+): MH+ 354.

### 4-[1-(5-Fluoro-2-methyl-benzenesulfonyl)-1H-pyrazol-3-yl]-quinoline (5)

Prepared from 4-(1H-pyrazol-3-yl)-quinoline and 5-fluoro-2-methyl-benzenesulfonylchloride using conditions described above.

¹H NMR (CDCl₃): 2.67 (3H, s), 6.85 (1H, d, J 2.6), 7.25-7.39 (2H, m), 7.51-7.60 (2H, m), 7.71-7.80 (1H, m), 7.92 (1H, dd, J 8.3 and 2.6), 8.15 (1H, d, J 8.8), 8.34 (1H, d, J 2.9), 8.39 (1H, d, J 8.3), 8.95 (1H, d, J 4.4).

MS(ESI+): MH+ 368.

### 4-[1-(3-Methoxy-benzenesulfonyl)-1H-pyrazol-3-yl]-quinoline (6)

Prepared from 4-(1H-pyrazol-3-yl)-quinoline and 3-methoxybenzenesulfonylchloride using conditions described above.

¹H NMR (CDCl₃): 3.88 (3H, s), 6.81 (1H, d, J 2.6), 7.20 (1H, dd, J 8.3 and 2.1), 7.48-7.80 (6H, m), 8.50 (1H, d, J 8.3), 8.28 (1H, d, J 2.6), 8.42 (1H, d, J 8.1), 8.95 (1H, d, J 4.5).

MS(ESI+): MH+ 366.

### 4-[1-(2,5-Dimethoxy-benzenesulfonyl)-1H-pyrazol-3-yl]-quinoline (7)

Prepared from 4-(1H-pyrazol-3-yl)-quinoline and 2,5-dimethoxybenzenesulfonylchloride using conditions described above.

¹H NMR (CDCl₃): 3.80 (3H, s), 6.79 (1H, d, J 2.8), 6.94 (1H, d, J 9.1), 7.18 (1H, dd, J 9.1 and 3.1), 7.47-7.75 (4H, m), 8.14 (1H, d, J 8.3), 8.35 (1H, d, J 8.4), 8.42 (1H, d, J 2.7), 8.93 (1H, d, J 4.4).

MS(ESI+): MH+ 396.

### 3-(3-Quinolin-4-yl-pyrazole-1-sulfonyl)-benzonitrile (8)

Prepared from 4-(1H-pyrazol-3-yl)-quinoline and 3-cyanobenzenesulfonyl chloride using conditions described above.

NMR: CDCl₃: 6.77 (1H, d, J 2.80, Ar),7.46 (1H, d, J 6.88, Ar), 7.48-7.52 (1H, m Ar), 7.60-7.68 (2 H, m, Ar), 7.82 (1H, dd, J 7.81 and 6.66, Ar), 8.09 (1H, d, J 8.32, Ar), 8.22 (1H, d, J 2.76, Ar), 8.26-8.37 (3H, m, Ar) and 8.88 (1H, d, J 4.43, Ar).

MS: (ESI+): MH+ 361.10

### 4-{1-[3-(5-Methyl-[1,2,4]oxadiazol-3-yl)-benzenesulfonyl]-1H-pyrazol-3-yl}-quinoline (9)

Prepared from 4-(1H-pyrazol-3-yl)-quinoline and 3-(5-methyl-[1,2,4]oxadiazol-3-yl)-benzenesulfonylchloride using conditions described above.

¹H NMR (CDCl₃): 2.68 (3H, s), 6.83 (1H, d, J 2.7), 7.51-7.78 (4H, m), 8.14-8.48 (5H, m), 8.83-8.85 (1H, m), 8.94 (1H, d, J 4.5).

MS(ESI+): MH+ 418.

### 4-{1-[3-(5-Methyl-[1,3,4]oxadiazol-2-yl)-benzenesulfonyl]-1H-pyrazol-3-yl}-quinoline (10)

Prepared from 4-(1H-pyrazol-3-yl)-quinoline and 3-(5-methyl-[1,3,4]oxadiazol-2-yl)-benzenesulfonylchloride using conditions described above.

¹H NMR (CDCl₃): 2.64 (3H, s), 6.83 (1H, d, J 2.7), 7.52-7.79 (4H, m), 8.16-8.43 (5H, m), 8.76-8.78 (1H, m), 8.95 (1H, d, J 4.4).

MS(ESI+): MH+ 418.

### Methyl 4-methoxy-3-(3-quinolin-4-yl-pyrazole-1-sulfonyl)-benzoate (11)

Prepared from 4-(1H-pyrazol-3-yl)-quinoline and methyl 3-chlorosulfonyl-4-methoxybenzoate using conditions described above.

¹H NMR (CDCl₃): 3.92 (3H, s), 3.94 (3H, s), 6.81 (1H, d, J 2.9), 7.05 (1H, d, J 8.8), 7.49-7.78 (3H, m), 8.14 (1H, d, J 8.2), 8.33-8.46 (3H, m), 8.88 (1H, d, J 2.2), 8.93 (1H, d, J 4.5).

MS(ESI+): MH+ 424.

### 4-{1-[3(2-Methyl-thiazol-4-yl)-benzenesulfonyl]-1H-pyrazol-3-yl}-quinoline (12)

Prepared from 4-(1H-pyrazol-3-yl)-quinoline and methyl 3-(2-methyl-thiazol-4-yl)-benzenesulfonylchloride using conditions described above.

¹H NMR (CDCl₃): 2.77 (3H, s), 6.80 (1H, d, J 2.6), 7.47-7.73 (5H, m), 8.06-8.23 (3H, m), 8.32 (1H, d, J 2.7), 8.39 (1H, d, J 9.5), 8.63 (1H, dd, J 1.8 and 1.8), 8.93 (1H, d, J 4.5).

MS(ESI+): MH+ 433.

### 3-{3-[6-Methoxy-7-(3-morpholin-4-yl-propoxy)-quinolin-4-yl]-pyrazole-1-sulfonyl}-benzonitrile (13)

Prepared from 6-methoxy-7-[3-(4-methyl-piperazin-1-yl)-propoxy]-4-(1H-pyrazol-3-yl)-quinoline and 3-cyanobenzenesulfonylchloride using conditions described above.

¹H NMR (CDCl₃): 1.52-1.59 (2H, m), 2.10-2.19 (2H, m), 2.47-2.65 (6H, m), 3.72-3.79 (2H, m), 3.97 (3H, s), 4.29 (2H, t, J 6.6), 6.89 (1H, d, J 2.9), 7.39 (1H, d J 4.6), 7.48 (1H, s), 7.72 (1H, dd, J 7.9 and 7.9), 7.93-7.95 (1H, m), 8.05 (1H, s), 8.32-8.40 (3H, m), 8.74 (1H, d, J 5.2).

MS(ESI+): MH+ 534.

### 3-(3-{6-Methoxy-7-[3-(4-methyl-piperazin-1-yl)-propoxy]-quinolin-4-yl}-pyrazole-1-sulfonyl)-benzonitrile (14)

Prepared from 6-methoxy-7-[3-(4-methyl-piperazin-1-yl)-propoxy]-4-(1H-pyrazol-3-yl)-quinoline and 3-cyanobenzenesulfonylchloride using conditions described above.

¹H NMR (CDCl₃): 1.52-1.58 (2H, m), 2.13 (2H, quintet, J 7.0), 2.30 (3H, s), 2.33-2.65 (10H, m), 3.96 (3H, s), 4.27 (2H, t, J 6.7), 6.89 (1H, d, J 2.9), 7.38 (1H, d, J 4.6), 7.47 (1H, s), 7.72 (1H, dd, J 7.9 and 7.9), 7.94 (1H, d, J 7.8), 8.03 (1H, s), 8.29-8.35 (3H, m), 8.74 (1H, d, J 4.7).

MS(ESI+): MH+ 547.

### 3-{3-[7-(3-Dimethylamino-propoxy)-6-methoxy-quinolin-4-yl]-pyrazole-1-sulfonyl}-benzonitrile (15)

Prepared from {3-[6-methoxy-4-(1H-pyrazol-3-yl)-quinolin-7-yloxy]-propyl}-dimethyl-amine and 3-cyanobenzenesulfonylchloride using conditions described above.

¹H NMR (CDCl₃): 2.12 (2H, quintet, J 7.2), 2.29 (6H, s), 2.53 (2H, t, J 7.2), 3.97 (3H, s), 4.27 (2H, t, J 7.2), 6.89 (1H, d, J 2.8), 7.38 (1H, d, J 4.7), 7.47 (1H, s), 7.72 (1H, d, J 7.9), 7.92-7.95 (1H, m), 8.04 (1H, s), 8.29-8.35 (3H, m), 8.74 (1H, d, J 4.6).

MS(ESI+): MH+ 492.

### 3-{3-[6-(3-Morpholin-4-yl-propoxy)-quinolin-4-yl]-pyrazole-1-sulfonyl}-benzonitrile (16)

Prepared from 6-(3-morpholin-4-yl-propoxy)-4-(1H-pyrazol-3-yl)-quinoline and 3-cyanobenzenesulfonylchloride using conditions described above.

¹H NMR (CDCl₃): 2.08 (2H, quintet, J 6.3), 2.45-2.62 (6H, m), 3.71-3.74 (4H, m), 4.16 (2H, t, J 6.3), 6.88 (1H, d, J 2.8), 7.42 (1H, dd, J 9.2 and 2.7), 7.48 (1H, d, J 4.5), 7.73 (1H, dd, J 7.8 and 7,8), 7.93-7.98 (2H, m), 8.05 (1H, d, J 9.2), 8.30 (1H, d, J 2.8), 8.34-8.37 (2H, m), 8.79 (1H, d, J 4.5).

MS(ESI+): MH+ 504.

### 3-(3-{6-[3-(4-Methyl-piperazin-1-yl]-propoxyl-quinolin-4-yl}-pyrazole-1-sulfonyl)-benzonitrile (17)

Prepared from 6-[3-(4-methyl-piperazin-1-yl)-propoxy]-4-(1H-pyrazol-3-yl)-quinoline and 3-cyanobenzenesulfonylchloride using conditions described above.

¹H NMR (CDCl₃): 2.08 (2H, quintet, J 6.3), 2.30 (3H, s), 2.45-2.68 (10H, m), 4.14 (2H, t, J 6.3), 6.88 (1H, d, J 2.7), 7.42 (1H, dd, J 9.2 and 2.8), 7.48 (1H, d, J 4.5), 7.71-7.76 (1H, m), 7.93-7.96 (1H, m), 8.05 (1H, d, J 9.2), 8.30 (1H, d, J 2.8), 8.35-8.38 (2H, m), 8.79 (1H, d, J 4.5).

MS(ESI+): MH+ 517.

### 3-{3-[6-(3-Dimethylamino-propoxy)-quinolin-4-yl]-pyrazole-1-sulfonyl}-benzonitrile (18)

Prepared from dimethyl-{3-[4-(1H-pyrazol-3-yl)-quinolin-6-yloxy]-propyl}-amine and 3-cyanobenzenesulfonylchloride using conditions described above.

¹H NMR (CDCl₃): 2.06 (2H, quintet, J 6.6), 2.30 (6H, s), 2.53 (2H, t, J 7.2), 4.14 (2H, t, 6.4), 6.88 (1H, d, J 2.6), 7.42 (1H, dd, J 9.2 and 2.7), 7.49 (1H, d, J 4.5), 7.71-7.76 (1H, m), 7.92-7.96 (2H, m), 8.05 (1H, d, J 9.2), 8.30 (1H, d, J 2.8), 8.36-8.39 (2H, m), 8.79 (1H, d, J 4.5).

MS(ESI+): MH+ 462.

### 4-[1-(2-Methyl-5-nitro-benzenesulfonyl)-4,5-dihydro1H-pyrazol-3-yl]-quinoline (19)

Reaction of 4-(4,5-dihydro-1H-pyrazol-3-yl)-quinoline with 2-methyl-5-nitrobenzene sulfonyl chloride as described above yielded the title compound

NMR: CDCl₃: 2.91 (3H, s, Me), 3.42 (2H, t, J 9.71, CH₂), 4.03 (2H, t, J 9.72, CH₂), 7.31 (1H, d, J 4.51, Ar), 7.52 (1H, d, J 8.43, Ar), 7.59-7.62 (1H, m, Ar), 7.73-7.76 (1H, m, Ar), 8.13 (1H, d, J 8.08, Ar), 8.32 (1H, dd, J 8.40 and 2.41, Ar), 8.81 (1H, d, J 8.20, Ar), 8.91 (1H, d, J 4.52, Ar) and 8.99 (1H, d, 2.41, Ar).

MS: (ESI+): MH+ 397.09

### (2-Methyl-5-nitro-phenyl)-(4-quinolin-4-yl-pyrimidin-2-yl)-amine (20)

To a solution of 3-dimethylamino-1-quinolin-4-yl-propenone (528 mg) (see Reference Example 5) and N-(2-methyl-5-nitro-phenyl)-guanidine nitric acid salt (1.20 g) in 2-methoxyethanol (1 mL) was added sodium hydroxide (94 mg) and the reaction mixture was heated at 125° C. for 16 h. After cooling to room temperature, the solvent was removed in vacuo and the residue was purified using column chromatography to give the title compound.

NMR: DMSO: 2.27 (3H, s, Me), 7.32-7.35 (1H, m, Ar), 7.45 (1H, d, J 8.32, Ar), 7.61-7.64 (1H, m, Ar), 7.69 (1H, d, J 4.38, Ar), 7.77-7.82 (1H, m, Ar), 8.13 (1H, d, J 8.40, Ar), 8.25 (1H, d, J 8.26, Ar), 8.77 (1H, d, J 5.00, Ar) and 9.04 (1H, d, J 4.37, Ar).

MS: (ESI+): MHMeCN+ 400.15

### 4-[2-(2-Methyl-5-nitro-phenylsulfanyl)-pyrimidin-4-yl]-quinoline (21)

To a mixture of sodium hydride (92 mg) in DMSO (7 mL) was added 4-quinolin-4-yl-pyrimidine-2-thiol (500 mg) and then 2-methyl-5-nitro-benzenediazonium tetrafluoroborate (531 mg) and the reaction stirred at room temperature for 16 h. The reaction was then quenched with water (20 mL) and extracted into ethyl acetate (20 mL) and the organic layer was washed with brine (3×20 mL), dried (MgSO₄), reduced in vacuo and purified by column chromatography to give the title compound.

NMR: CDCl₃: 2.58 (3H, s, Me), 7.36 (1H, d, J 5.04, Ar), 7.43-7.49 (3H, m, Ar), 7.73-7.76 (1H, m, Ar), 8.02 (1H, d, J 8.11, Ar), 8.18 (2H, dd, J 2.31 and 8.42, Ar), 8.61 (1H, d, J 2.40, Ar), 8.67 (1H, d, 5.04, Ar) and 9.00 (1H, d, J 4.39, Ar).

MS: (ESI+): MH+ 375.07

### 4-[2-(2-Methyl-5-nitro-phenylsulfanyl)-thiazol-4-yl]-quinoline (22)

Reaction between 4-quinolin-4-yl-thiazole-2-thiol and 2-methyl-5-nitro-benzenediazonium tetrafluoroborate in DMSO with sodium hydride, as described above, yielded the title compound.

NMR: CDCl₃: 2.65 (3H, s, Me), 7.51-7.61 (4H, m, Ar), 7.74 (1H, apparent triplet, J 6.98, Ar), 8.18-8.21 (2H, m, Ar), 8.30 (1H, d, J 7.90, Ar), 8.57 (1H, d, J 2.36, Ar) and 8.96 (1H, d, J 4.44, Ar).

MS: (ESI+): MH+ 380.10

### 4-(1-Methanesulfonyl-1H-pyrazol-3-yl)-quinoline-6-carbonitrile (23)

A mixture of 4-(1H-pyrazol-3-yl)-quinoline-6-carbonitrile (50 mg) and methanesulfonyl chloride (0.5 mL) in pyridine (4 mL) was stirred at room temperature for 16 h. The reaction was then poured into ice water (20 mL) and the solid filtered and washed with water and diethyl ether to give the title compound.

NMR: DMSO: 3.73 (3H, s, Me), 7.36 (1H, d, J 2.87, Ar), 8.05 (1H, d, J, 4.52, Ar), 8.14 (1H, dd, J 8.77 and 1.83, Ar), 8.28 (1H, d, J 2.66, Ar), 8.58 (1H, d, J 2.66, Ar), 9.18 (1H, d, J 4.52, Ar) and 9.28 (1H, d, J 1.60, Ar).

MS: (ESI+): MH+ 299.10

### Example 2

### Biological Testing

Compounds of the invention, prepared as described in the preceding Examples, were submitted to the following assay:

In a final volume of 25 ul, Met (h) (5-10 mU) is incubated with 8 mM MOPS pH7.0, 0.2 mM EDTA, 250 uM KKKSPGEYVNIEFG, 10 mM Mg Acetate, 45 uM ATP (KM), and [y33p-ATP] (approximately specific activity 500 cpm/pmol concentration is required). The reaction is initiated by addition of the Mg/ATP mix. After incubation of 40 minutes at room temperature, the reaction is stopped by addition of 5 ul of 3% phosphoric acid solution. 10 ul of the reaction is then spotted onto a P30 filtermat and washed 3 times for 5 minutes in 75 mM phosphoric acid and once in methanol prior to drying and scintillation counting.

All compounds are prepared in 100% DMSO and screened at 0.5 uM and 10 uM.

Several compounds demonstrated ≧90% inhibition of the enzyme at 0.5 uM

### Example 3

### Tablet Composition

Tablets, each weighing 0.15 g and containing 25 mg of a compound of the invention are manufactured as follows:

### Composition for 10,000 Tablets

Active compound (250 g)
Lactose (800 g)
Corn starch (415 g)
Talc powder (30 g)
Magnesium stearate (5 g)

The active compound, lactose and half of the corn starch are mixed. The mixture is then forced through a sieve 0.5 mm mesh size. Corn starch (10 g) is suspended in warm water (90 ml). The resulting paste is used to granulate the powder. The granulate is dried and broken up into small fragments on a sieve of 1.4 mm mesh size. The remaining quantity of starch, talc and magnesium is added, carefully mixed and processed into tablets.

### Example 4

### Injectable Formulation

### Formulation A

| | |
|---|---|
| Active compound | 200 mg |
| Hydrochloric Acid Solution 0.1M or | 4.0 to 7.0 |
| Sodium Hydroxide Solution 0.1M q.s. to pH | |
| Sterile water q.s. to | 10 ml |

The compound of the invention is dissolved in most of the water (35° 40° C.) and the pH adjusted to between 4.0 and 7.0 with the hydrochloric acid or the sodium hydroxide as appropriate. The batch is then made up to volume with water and filtered through a sterile micropore filter into a sterile 10 ml amber glass vial (type 1) and sealed with sterile closures and overseals.

### Formulation B

| | | |
|---|---|---|
| Active Compound | 125 | mg |
| Sterile, Pyrogen-free, pH 7 Phosphate | 25 | ml |
| Buffer, q.s. to | | |
| Active compound | 200 | mg |
| Benzyl Alcohol | 0.10 | g |
| Glycofurol 75 | 1.45 | g |
| Water for injection q.s to | 3.00 | ml |

The active compound is dissolved in the glycofurol. The benzyl alcohol is then added and dissolved, and water added to 3 ml. The mixture is then filtered through a sterile micropore filter and sealed in sterile 3 ml glass vials (type 1).

### Example 5

### Syrup Formulation

| | | |
|---|---|---|
| Active compound | 250 | mg |
| Sorbitol Solution | 1.50 | g |
| Glycerol | 2.00 | g |
| Sodium benzoate | 0.005 | g |
| Flavour | 0.0125 | ml |
| Purified Water q.s. to | 5.00 | ml |

The compound of the invention is dissolved in a mixture of the glycerol and most of the purified water. An aqueous solution of the sodium benzoate is then added to the solution, followed by addition of the sorbitol solution and finally the flavour. The volume is made up with purified water and mixed well.

1. A compound which is a fused pyridine of formula (I): 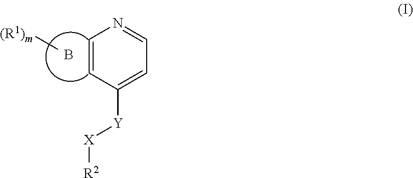
wherein
B is an aryl or heteroaryl ring;
each R¹, which are the same or different when m is greater than 1, is selected from H, halogen, CN, OR³, alkyl, alkenyl, alkynyl, CF₃, —O(C(R³)₂)ₙNR⁴R⁵, —NR³(C(R³)₂)ₙNR⁴R⁵, —NR⁴R⁵, —CONR⁴R⁵, —SO₂NR⁴R⁵, NO₂, —S(O)ₚR³, —CO₂R³, —NR³COR³, —NR³SO₂R³ and R⁶,
m is 0, 1 or 2;
Y is a heteroaryl or dihydroheteroaryl ring, or a group —C≡C—C(R′)₂—;
X is selected from —O—, —S—, —SO—, —SO₂—, —NR³—, —NR³SO—, —NR³SO₂—, —N(SO₂R³)—, —CO—, —CONR³—, —NR³CO—, —NR³CONR³—, —NR³CS— and —NR³CSNR³, provided that X is other than —NH— when Y is a pyrimidine ring;
R² is selected from aryl which is unsubstituted or substituted, heteroaryl which is unsubstituted or substituted, C₁-C₆ alkyl which is unsubstituted, and C₂-C₆ alkenyl and C₂-C₆ alkynyl which are unsubstituted or substituted;
R³ is selected from H, C₁-C₆ alkyl, C₂-C₆ alkenyl and C₂-C₆ alkynyl;
R⁴ and R⁵, which are the same or different, are each selected from H, C₁-C₆ alkyl, C₁-C₆ alkenyl and C₁-C₆ alkynyl, or R⁴ and R⁵ together with the N atom to which they are attached form a 5- or 6-membered heterocyclic ring containing 0, 1 or more additional heteroatoms selected from N, O and S;
n is 2 or 3;
p is 1 or 2;
R⁶ is an aryl or heteroaryl ring which is unsubstituted or substituted; and
R′ is H or C₁-C₆ alkyl;
or a pharmaceutically acceptable salt thereof.

2. A compound according to claim 1 wherein the fused pyridine is of the following formula (Ia): 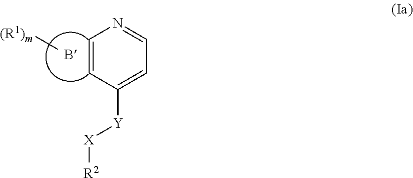
wherein
B′ is selected from a benzene, pyridine, pyrrole and pyrazole ring; and
R¹, R², X, Y and m are as defined in claim 1.

3. The compound according to claim 1 wherein X is selected from —S—, —SO₂—, —CO—, —CONR³—, —NR³CO—, —NR³CONR³—, and —N(SO₂R³)—, wherein R³ is H or C₁-C₆ alkyl.

4. The compound according to claim 1 wherein Y is a ring selected from pyrazole, pyrimidine, thiazole, oxazole, pyrrole, dihydropyrazole, thiophene, furan and benzene.

5. The compound according to claim 1 in which ring B is a benzene ring.

6. A compound which is selected from:
4-[1-(2-Methyl-5-nitro-benzenesulfonyl)-1H-pyrazol-4-yl]-quinoline;
4-[1-(2-Methyl-5-nitro-benzenesulfonyl)-1H-pyrazol-3-yl]-quinoline;
4-Methyl-3-(3-quinolin-4-yl-pyrazole-1-sulfonyl)-benzonitrile;
4-[1-(3-Fluoro-benzenesulfonyl)-1H-pyrazol-3-yl]-quinoline;
4-[1-(5-Fluoro-2-methyl-benzenesulfonyl)-1H-pyrazol-3-yl]-quinoline;
4-[1-(3-Methoxy-benzenesulfonyl)-1H-pyrazol-3-yl]-quinoline;
4-[1-(2,5-Dimethoxy-benzenesulfonyl)-1H-pyrazol-3-yl]-quinoline;
3-(3-Quinolin-4-yl-pyrazole-1-sulfonyl)-benzonitrile;
4-{1-[3-(5-Methyl-[1,2,4]oxadiazol-3-yl)-benzenesulfonyl]-1H-pyrazol-3-yl}-quinoline;
4-{1-[3-(5-Methyl-[1,3,4]oxadiazol-2-yl)-benzenesulfonyl]-1H-pyrazol-3-yl}quinoline;
Methyl 4-methoxy-3-(3-quinolin-4-yl-pyrazole-1-sulfonyl)-benzoate;
4-{1-[3-(2-Methyl-thiazol-4-yl)-benzenesulfonyl]-1H-pyrazol-3-yl}-quinoline;
3-{3-[6-Methoxy-7-(3-morpholin-4-yl-propoxy)-quinolin-4-yl]-pyrazole-1-sulfonyl}-benzonitrile;
3-(3-{6-Methoxy-7-[3-(4-methyl-piperazin-1-yl)-propoxy]-quinolin-4-yl}-pyrazole-1-sulfonyl)-benzonitrile;
3-{3-[7-(3-Dimethylamino-propoxy)-6-methoxy-quinolin-4-yl]-pyrazole-1-sulfonyl}-benzonitrile;
3-{3-[6-(3-Morpholin-4-yl-propoxy)-quinolin-4-yl]-pyrazole-1-sulfonyl}-benzonitrile;
3-(3-{6-[3-(4-Methyl-piperazin-1-yl)-propoxy]-quinolin-4-yl}-pyrazole-1-sulfonyl)-benzonitrile;
3-{3-[6-(3-Dimethylamino-propoxy)-quinolin-4-yl]-pyrazole-1-sulfonyl}-benzonitrile;
4-[1-(2-Methyl-5-nitro-benzenesulfonyl)-4,5-dihydro-1H-pyrazol-3-yl]-quinoline;
4-[2-(2-Methyl-5-nitro-phenylsulfanyl)-pyrimidin-4-yl]-quinoline;
4-[2-(2-Methyl-5-nitro-phenylsulfanyl)-thiazol-4-yl]-quinoline; and
4-(1-Methanesulfonyl-1H-pyrazol-3-yl)-quinoline-6-carbonitrile;
and the pharmaceutically acceptable salts thereof.

7. A pharmaceutical composition which comprises a pharmaceutically acceptable carrier or diluent and, as an active ingredient, a compound of claim 1.

8. A pharmaceutical composition which comprises a pharmaceutically acceptable carrier or diluent and, as an active ingredient, a compound of claim 2.

9-11. (canceled)

12. A method of treating a patient in need of an inhibitor of c-Met, which method comprises administering to the patient a compound which is is a fused pyridine of formula (I): 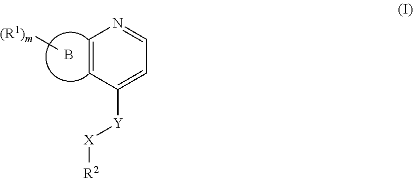
wherein
B is an aryl or heteroaryl ring;
each R¹, which are the same or different when m is greater than 1, is selected from H, halogen, CN, OR³, alkyl, alkenyl, alkynyl, CF₃, —O(C(R³)₂)ₙNR⁴R⁵, —NR³(C(R³)₂)ₙNR⁴R⁵, —NR⁴R⁵, —CONR⁴R⁵, —SO₂NR⁴R⁵, NO₂, —S(O)ₚR³, —CO₂R³, —NR³COR³, —NR³SO₂R³ and R⁶;
m is 0, 1 or 2;
Y is a heteroaryl, dihydroheteroaryl or aryl ring, or a group —C≡C—C(R′)₂—;
X is selected from —C(R³)₂—, —O—, —S—, —SO—, —SO₂—, —NR³—, —NR³SO—, —NR³SO₂—, —N(SO₂R³)—, —CO—, —CONR³—, —NR³CO—, —NR³CONR³—, —CSNR³—, —NR³CS— and —NR³CSNR³;
R² is selected from aryl which is unsubstituted or substituted, heteroaryl which is unsubstituted or substituted, C₁-C₆ alkyl, C₂-C₆ alkenyl and C₂-C₆ alkynyl;
R³ is selected from H, C₁-C₆ alkyl, C₂-C₆ alkenyl and C₂-C₆ alkynyl;
R⁴ and R⁵, which are the same or different, are each selected from H, C₁-C₆ alkyl, C₁-C₆ alkenyl and C₁-C₆ alkynyl, or R⁴ and R⁵ together with the N atom to which they are attached form a 5- or 6-membered heterocyclic ring containing 0, 1 or more additional heteroatoms selected from N, O and S;
n is 2 or 3;
p is 1 or 2;
R⁶ is an aryl or heteroaryl ring which is unsubstituted or substituted; and
R′ is H or C₁-C₆ alkyl;
or a pharmaceutically acceptable salt thereof.

13. A method according to claim 12 wherein the patient is suffering from a disease or disorder selected from the group consisting of cancer, cardiovascular disease, an immunological disorder and an ocular disorder.

14. The compound according to claim 2 wherein X is selected from —S—, —SO₂—, —CO—, —CONR³—, —NR³CO—, —NR³CONR³—, and —N(SO₂R³)—, wherein R³ is H or C₁-C₆ alkyl.

15. The compound according to claim 2 wherein Y is a ring selected from pyrazole, pyrimidine, thiazole, oxazole, pyrrole, dihydropyrazole, thiophene, furan and benzene.

16. The compound according to claim 2 in which ring B is a benzene ring.

17. The method of claim 12 wherein the fused pyridine is of the following formula (Ia): 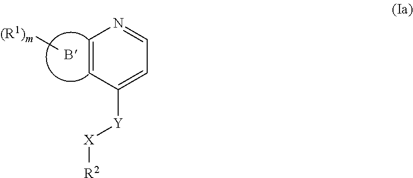
wherein
B′ is selected from a benzene, pyridine, pyrrole and pyrazole ring; and
R¹, R², X, Y and m are as defined in claim 12.
